(19) United States
(12) Patent Application Publication
(10) Pub. No.: US 2025/0152216 A1
(43) Pub. Date: May 15, 2025

(54) IMPLANT WITH ADJUSTABLE PLATES FOR MINIMALLY INVASIVE ORTHOGNATHIC SURGERY AND ITS USE

(71) Applicant: TRAUMEC TECNOLOGIA E IMPLANTES ORTOPÉDICOS IMPORTAÇÃO E EXPORTAÇÃO LTDA, Rio Claro (BR)

(72) Inventor: CARLOS CÉSAR CARITÁ, Rio Claro (BR)

(21) Appl. No.: 18/416,539

(22) Filed: Jan. 18, 2024

(30) Foreign Application Priority Data
Nov. 14, 2023 (BR) 1020230238882

(51) Int. Cl. A61B 17/80; A61B 17/56

(52) U.S. Cl. CPC A61B 17/8071; A61B 17/8057; A61B 2017/564

(57) ABSTRACT

Adjustable plates for minimally invasive orthognathic surgery are made of pure titanium, with easy adaptation and excellent mechanical resistance, biocompatible, pre-molded, anatomical designers, low profile, wide range of models and rigid fixation system. Consequently, the plates generate less surgical trauma, less risk of infection, less blood loss and smaller scars; the field of application of the invention being aimed at the area of medicine and restorative and/or aesthetic dentistry.

### FIELD OF APPLICATION

This is a patent application for the invention of new adjustable plates for minimally invasive orthognathic surgery; these plates are made of pure titanium, with easy adaptation and excellent mechanical resistance, biocompatible, pre-molded, anatomical designs, low profile, wide range of models and rigid fixation system. Consequently, they generate less surgical trauma, less risk of infection, less blood loss and smaller scars; the field of application of the invention being aimed at the area of medicine and restorative and/or aesthetic dentistry.

### BRIEF SUMMARY OF THE INVENTION

This invention presents innovative plates, such as: chin plates with a reduced design; mandibular plates with angled posterior holes and a reduced design; maxillary plates segmented in the upper portion and restructured in the central region; and pre-molded straight and “L” plates. The use of the plates of this invention means that, unlike traditional techniques, surgical accesses are minimal and with less tissue displacement, reducing surgical time and providing a faster and more satisfactory post-operative period. In other words, according to the invention, plates are presented with a reduced design, angled posterior holes, restructured central region, greater rigidity and pre-molded; these are some of the differentials offered by the new osteosynthesis cases with the plates presented here.

### PRIOR ART

The document BR112022008210-0, published on Jul. 12, 2022, in the name of Travis Simpson and others, with union priority U.S. 62/926,965, entitled ORTHOGNATHIC SURGICAL IMPLANT SET WITH PRE-OSTEOTOMY AND POST-OSTEOTOMY ALIGNMENT ELEMENTS, is known in prior art, which essentially describes an implant assembly and its method of use, used in computer-assisted orthognathic surgery to adequately reposition a mobile bone segment separated by osteotomy from a malformed mandible, maxilla or chin, the implant assembly comprising fixation bone plates, at least one removable alignment plate for correct pre-osteotomy placement of the implant assembly in the mandible, maxilla or chin, at least one removable post-osteotomy alignment plate for proper positioning of the movable bone segment in relation to the mandible, maxilla or chin, and a removable cutting guide for guiding the osteotomy saw during separation of the movable bone segment from the mandible, maxilla or chin.

The aforementioned document further describes, as an object of the invention, an improved patient-specific orthognathic surgical implant assembly and a method of use that solves this problem by providing such an assembly with an increased amount of contact surface areas so that the proper location of the bone guide and plates is more easily achieved. It is an object of this invention to provide such an improved orthognathic surgical implant assembly with the assembly having one or more removable pre-osteotomy alignment plate elements for proper pre-osteotomy positioning of the fixation bone plate(s) in the non-mobile or base bone segment and one or more removable post-osteotomy alignment plate elements for proper post-osteotomy positioning of the fixation bone plate(s) in the mobile or detached bone segment(s), as well as the method of use of such an implant assembly. It is an additional objective to provide, in some modalities, such an implant assembly with a cutting guide for marking or performing the osteotomy.

Document BR112012026997-6, published on Oct. 3, 2017, on behalf of SYNTHES GMBH (CH), filed via PCT and started the national phase on Oct. 22, 2012, entitled “IMPLANT AND METHOD OF CUSTOMIZING A PRE-FORMED IMPLANT”, essentially describes an implant for use in an orthognathic mandibular surgery may include a longitudinal plate member and a plurality of pre-configured guides coupled to a plate member. The longitudinal plate member is pre-curved to match the post-operative shape of the mandible; and the guides are pre-configured to align the plate member with the mandible when the implant is positioned against the mandible after the mandible has been separated.

Document BR112012026703-5, published on Oct. 10, 2017, in the name of SYNTHES GMBH (CH), granted a patent on Apr. 7, 2020, entitled ORTHOGNATHIC IMPLANT AND METHODS OF USE, describes an implant for use in an orthognathic surgery of a maxilla may include a longitudinal plate member and a plurality of pre-formed fingers extending from an upper edge of the plate member. The longitudinal plate member is pre-formed to match the pre-operative shape of a maxilla; and the fingers are pre-formed to match the shape of the maxilla. The fingers may be pre-formatted to match both the preoperative shape and the postoperative shape of the maxilla.

The prior art documents listed above, as well as the existing and consolidated plates on the market, have larger dimensions, are not anatomical and have more robust profiles, so they need to be shaped manually during use, consequently causing greater trauma to patients, longer procedure and recovery times and greater risks.

### OBJECTIVES OF THE INVENTION

The objective of the invention is to provide an implant through adjustable plates for orthognathic surgery, intended in the oral and maxillofacial segment for the correction of dentofacial deformities. Unlike traditional techniques, the use of these plates enables minimal surgical access and less tissue detachment, reducing surgical time and providing a faster and more satisfactory post-operative period.

Another objective of the invention comprises a range of plates for orthognathic surgeries, such as: chin plates with a reduced design; mandibular plates with angled posterior holes and a reduced design; maxillary plates segmented in the upper portion and restructured in the central region and; pre-molded straight and “L” plates; so that these plates can be used in minimally invasive orthognathic surgeries, having the following fundamental characteristics: being made of pure titanium, easy to adapt and with excellent mechanical resistance, biocompatible, pre-molded, anatomical designers, low profile, wide range of models and rigid fixation system.

As a result of the new range of plates, through adaptations in conformation, when compared to the plates on the market, in relation to the bone anatomy, applications and techniques employed, it is another objective of the present invention to facilitate operative techniques, as well as to reduce the time of patient exposure.

### GENERAL DESCRIPTION OF THE INVENTION

THE IMPLANT WITH ADJUSTABLE PLATES FOR MINIMALLY INVASIVE ORTHOGNATHIC SURGERY AND ITS USE, the subject of this patent application, comprises a system made up of a range of implants (plates) intended for use in minimally invasive surgery. These plates are divided into chin plates with a reduced design, mandibular plates with angled posterior holes and a reduced design, maxillary plates segmented in the upper portion and restructured in the central region, maxillary plates segmented in the upper portion and restructured in the central region and; pre-molded straight and L-shaped plates. These implants or plates have particular characteristics that are linked to various factors. The fact that they are made of pure titanium is not only linked to the issue of mechanical resistance, but also to the fact that these pre-molded plates are biocompatible, have anatomical designs, a low profile and this wide range of plates has a rigid fixation system. As previously mentioned, this leads to less surgical trauma, faster recovery, less risk of infection, less blood loss and equally smaller scars.

### DESCRIPTION OF DESIGNS

The invention will be described below in all its characteristics, and for a better understanding, references will be made to the attached drawings in which they are represented:

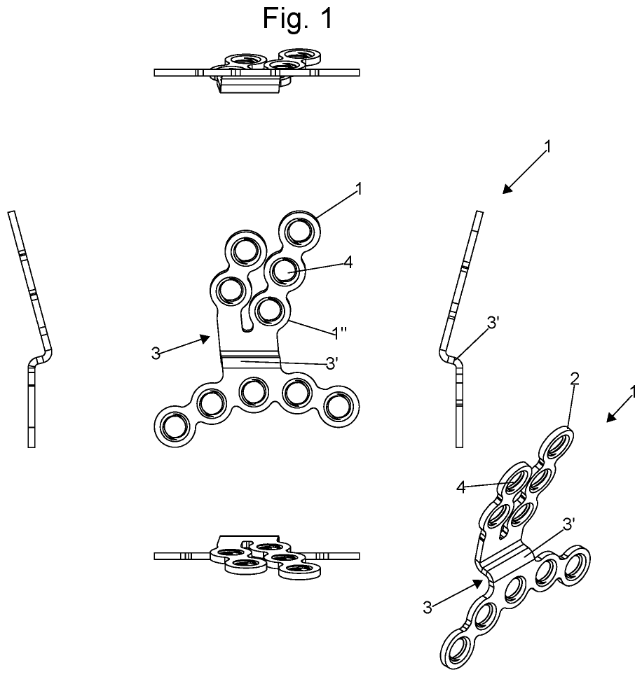

FIG. 1: Front, right side, left side, top, bottom and angled views of a Lefort plate type implant;

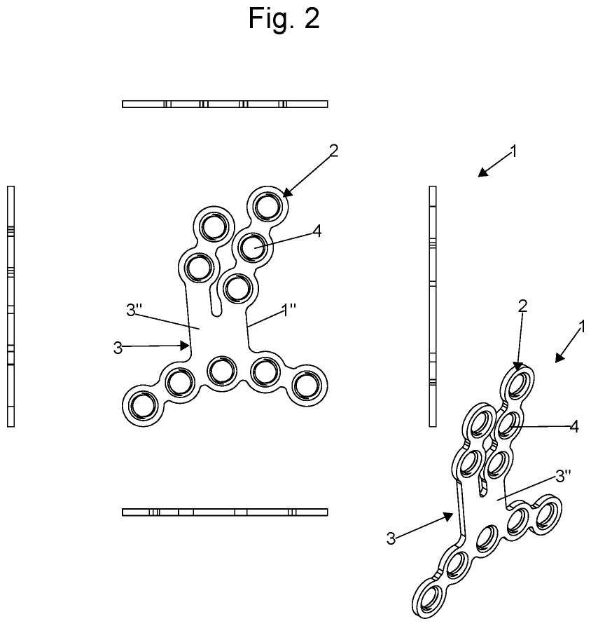

FIG. 2: Front, right side, left side, top, bottom and angled views of a Lefort plate implant, in a constructive variation;

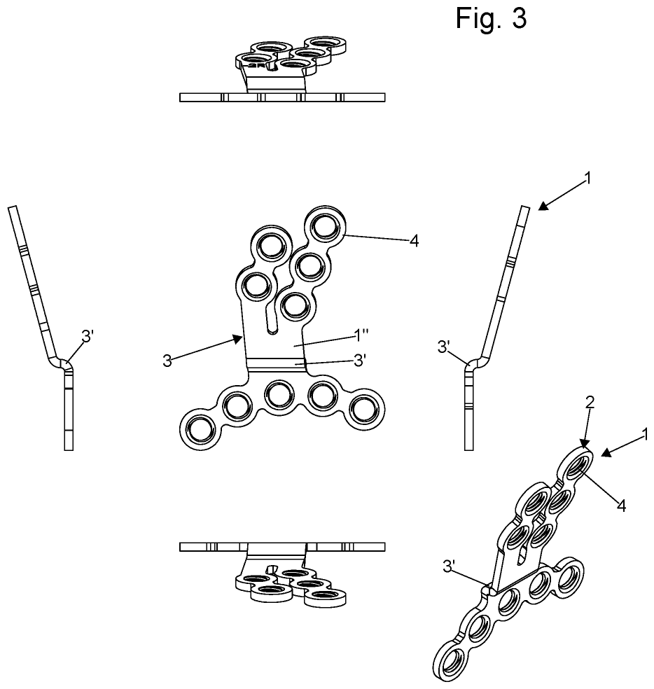

FIG. 3: Front, right side, left side, top, bottom and angled views of a Lefort plate implant, in a second constructive variation;

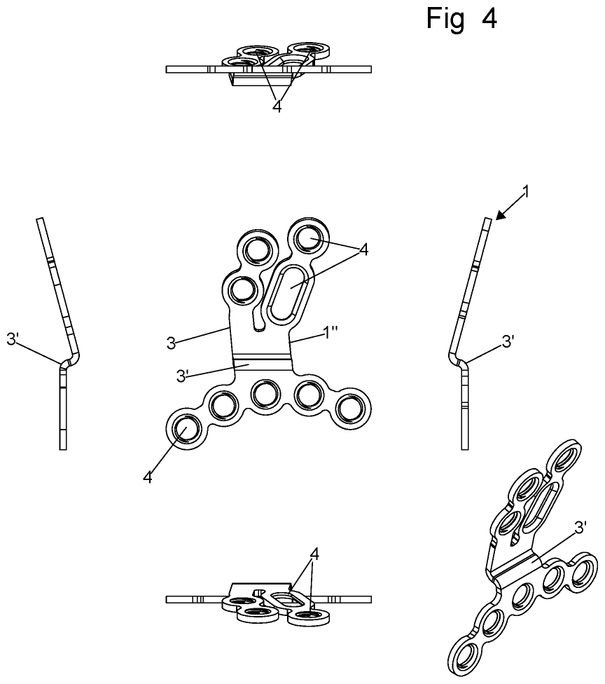

FIG. 4: Front, right side, left side, top, bottom and angled views of a Lefort plate implant, in a third constructive variation;

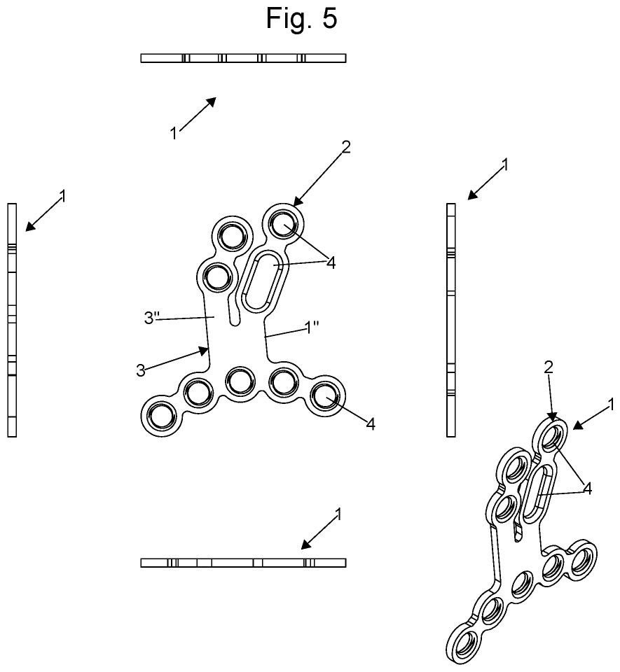

FIG. 5: Front, right side, left side, top, bottom and angled views of a Lefort plate implant, in a fourth constructive variation;

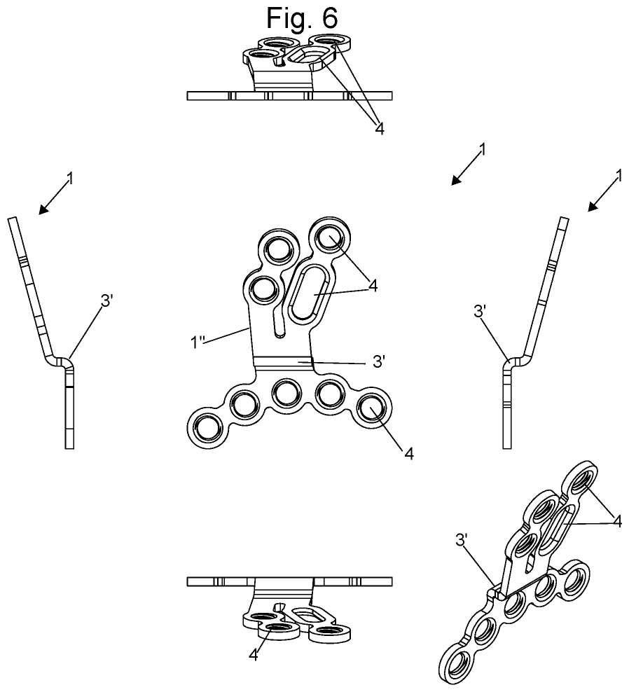

FIG. 6: Front, right side, left side, top, bottom and angled views of a Lefort plate implant, in a fifth constructive variation;

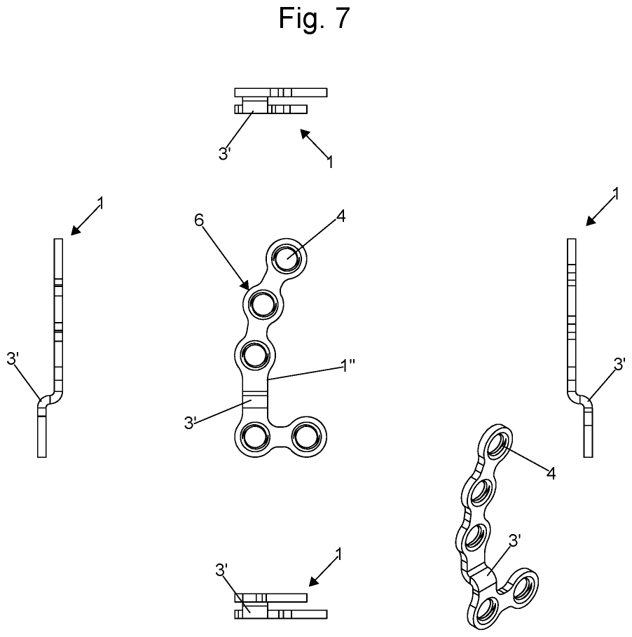

FIG. 7: Front, right side, left side, top, bottom and angled views of a Lefort plate implant, for the maxilla, in a sixth constructive variation;

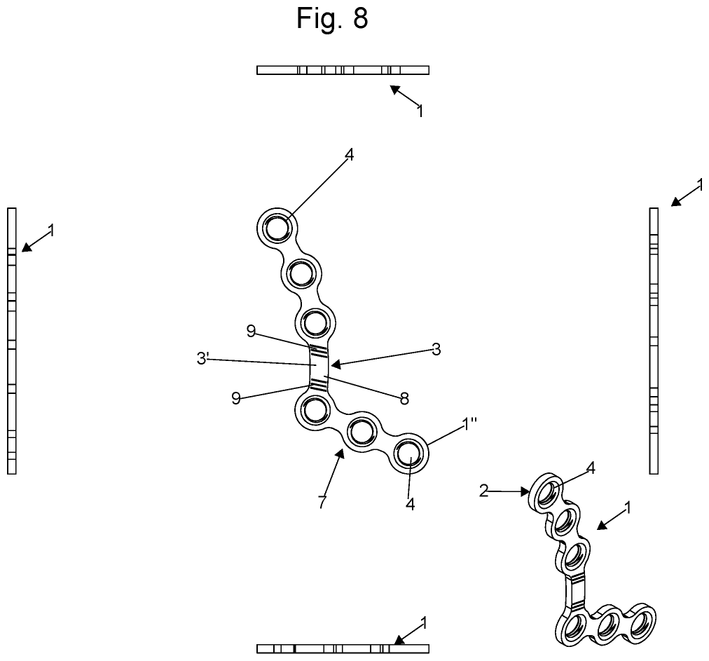

FIG. 8: Front, right side, left side, top, bottom and angled views of a Lefort plate implant, in a seventh constructive variation;

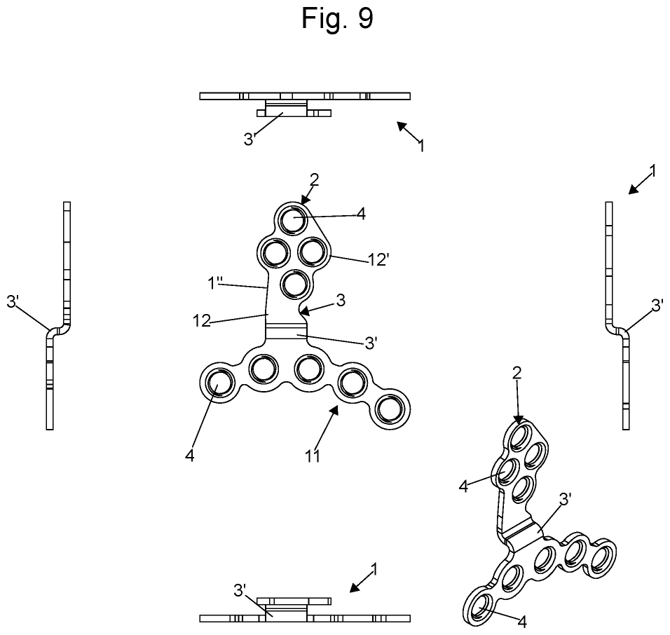

FIG. 9: Front, right side, left side, top, bottom and angled views of a Lefort plate implant, in an eighth constructive variation;

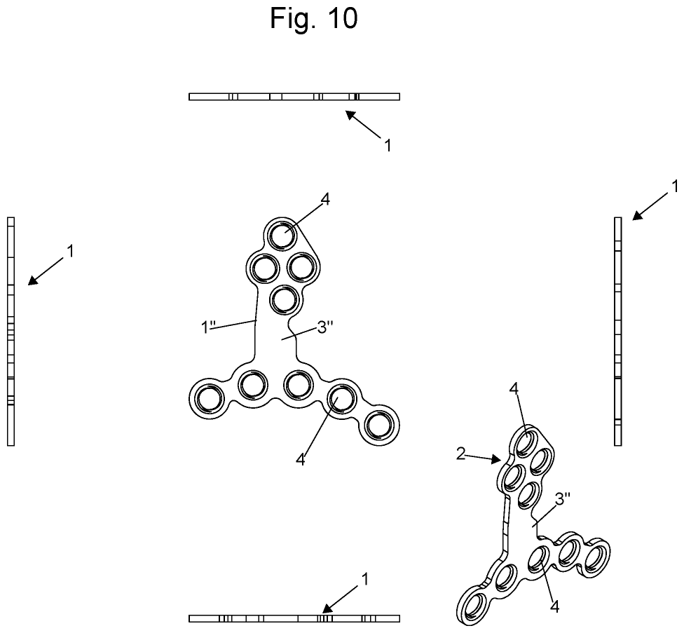

FIG. 10: Front, right side, left side, top, bottom and angled views of a Lefort plate implant, in a ninth constructive variation;

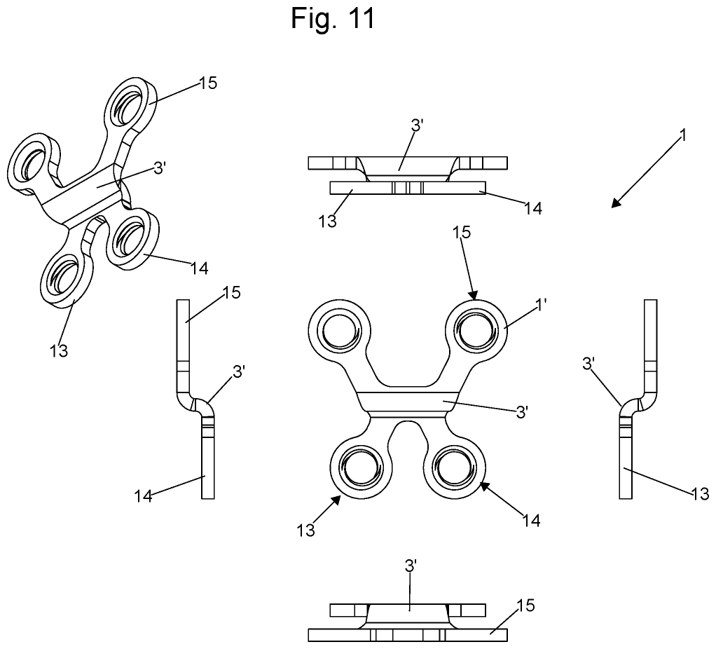

FIG. 11: Front, right side, left side, top, bottom and angled views of a chin advancement plate implant;

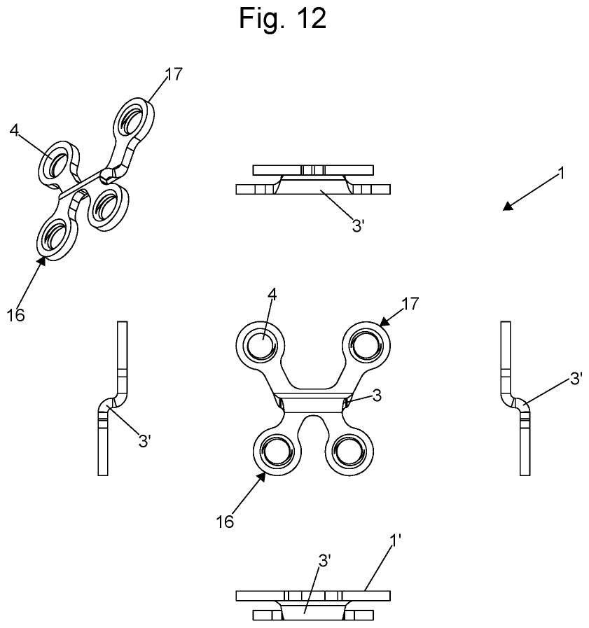

FIG. 12: Front, right side, left side, top, bottom and angled views of a chin advancement plate implant, in a first constructive variation;

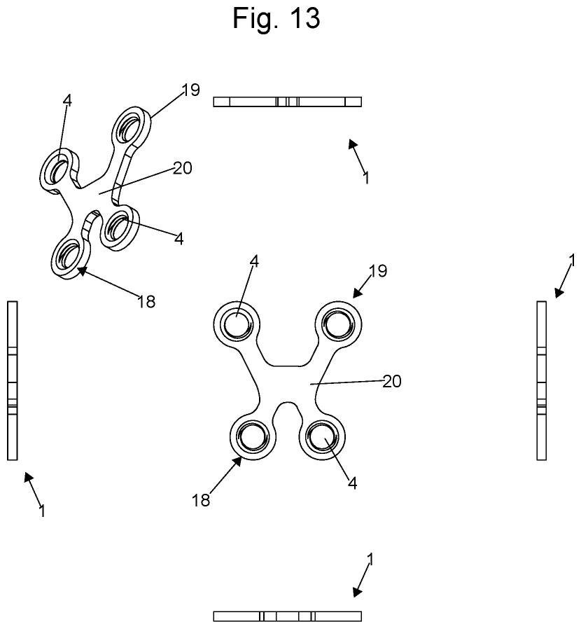

FIG. 13: Front, right side, left side, top, bottom and angled views of a chin advancement plate implant, in a second constructive variation;

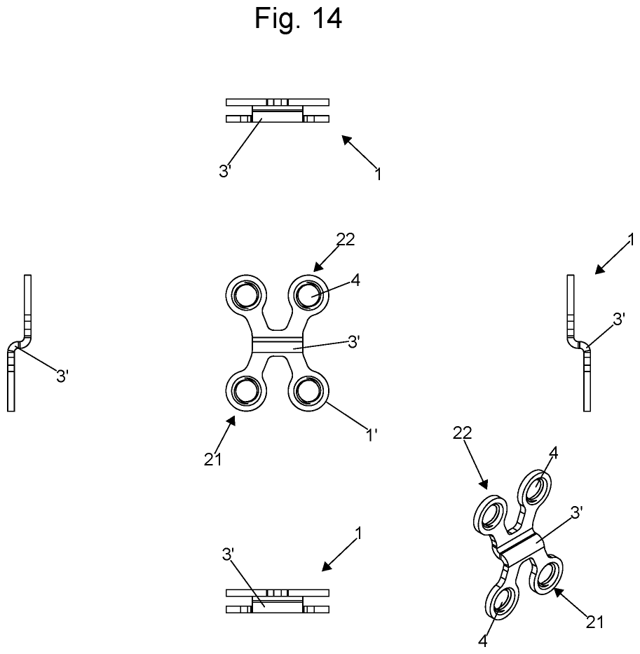

FIG. 14: Front, right side, left side, top, bottom and angled views of a chin advancement plate implant in a third constructive variation;

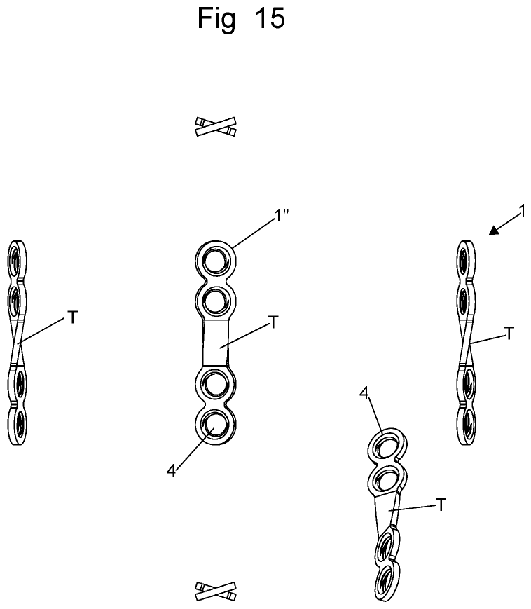

FIG. 15: Front, right side, left side, top, bottom and angled views of a straight, pre-molded-Lefort maxilla plate implant;

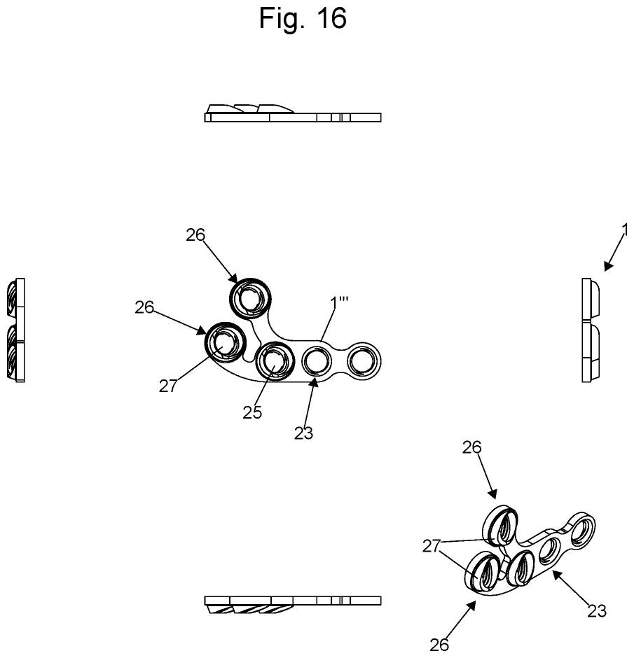

FIG. 16: Front, right side, left side, top, bottom and angled views of a mandible plate implant;

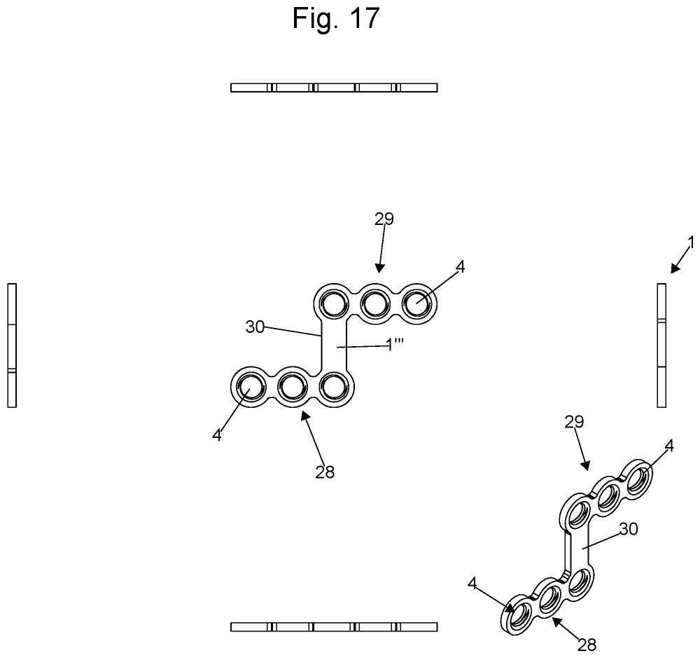

FIG. 17: Front, right side, left side, top, bottom and angled views of a mandible plate implant, in a constructive variation;

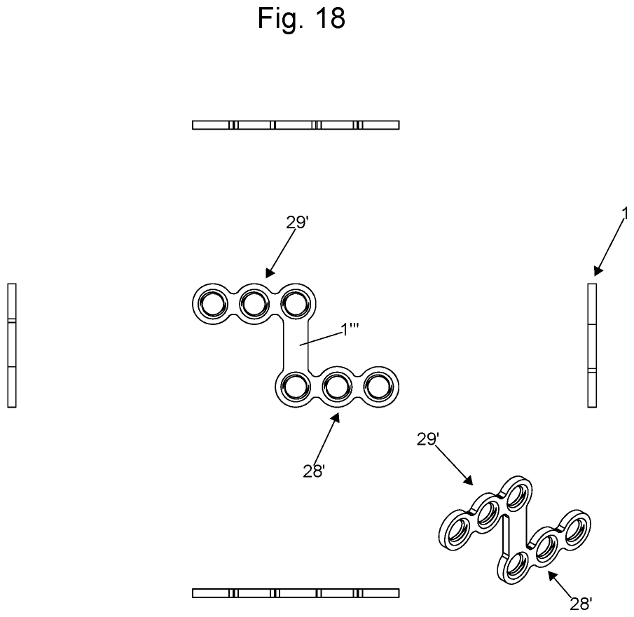

FIG. 18: Front, right side, left side, top, bottom and angled views of a mandible plate implant, in a second constructive variation;

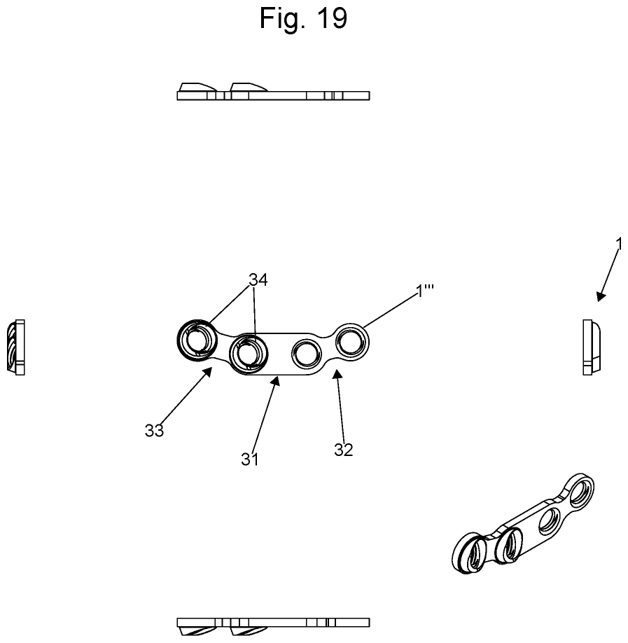

FIG. 19: Front, right side, left side, top, bottom and angled views of a mandible plate implant, in a third constructive variation;

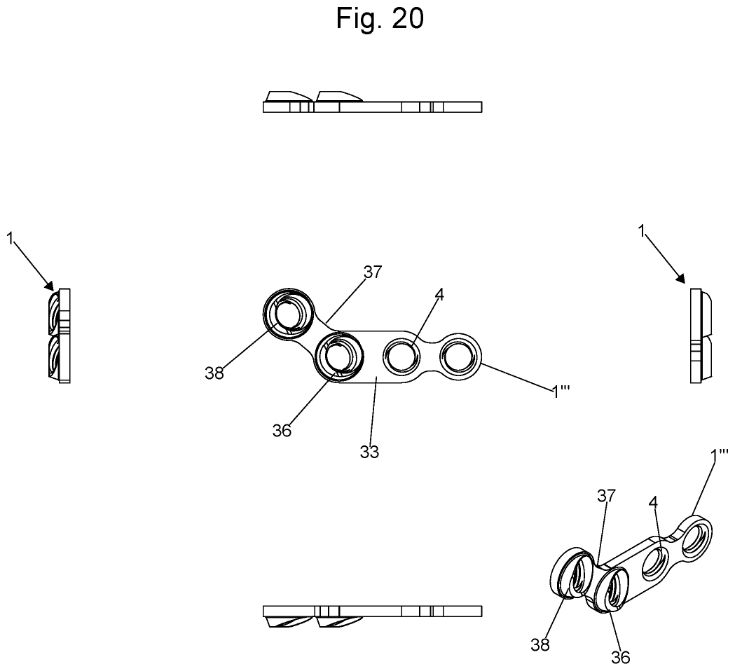

FIG. 20: Front, right side, left side, top, bottom and angled views of a mandible plate implant, in a fourth constructive variation;

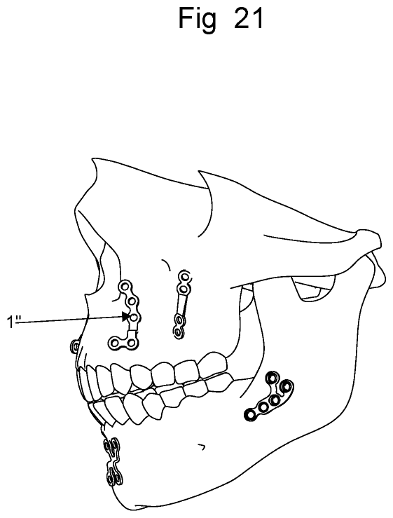

FIG. 21: It illustrates, in an oral and maxillofacial model, the application of the implants (plates) of the invention, in a Lefort-type example of use;

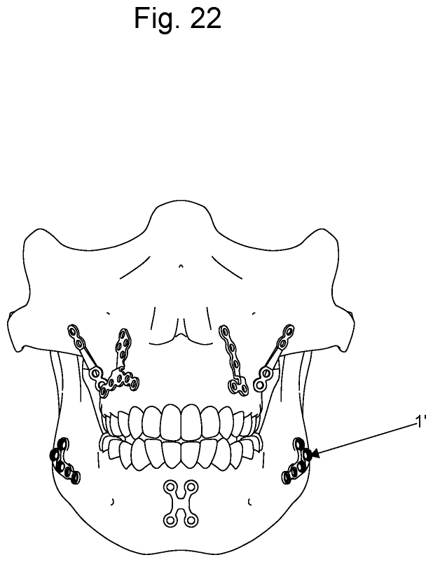

FIG. 22: It illustrates, in an oral and maxillofacial model, the application of the implants (plates) of the invention, in a chin-type example of use;

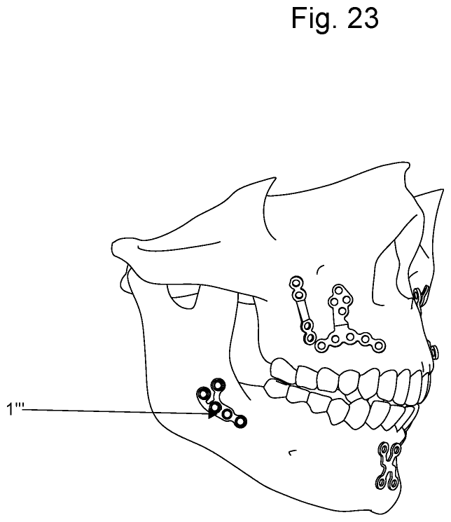

FIG. 23: It illustrates, in an oral and maxillofacial model, the application of the implants (plates) of the invention, in a sagittal-mandible type example of use.

### DETAILED DESCRIPTION OF THE INVENTION

THE IMPLANT WITH ADJUSTABLE PLATES FOR MINIMALLY INVASIVE ORTHOGNATHIC SURGERY AND ITS USE, the subject of this patent application, comprises a range of implants (plates) (1), made of titanium, intended for use in minimally invasive orthognathic surgery, made up of chin-type plates (1′), Lefort-type (1″) maxillary plates and sagittal-type (1″) mandibular plates, all with a reduced design, which are segmented in the upper portion and restructured in the central region (3) and pre-molded straight and “L” plates (1″)—including Lefort; these plates are presented in the form of a case, so that the surgeon can use them fully in surgical procedures for correcting the mandible, maxillary regions and chin, as their pre-molded characteristics and reduced design (compared to thick conventional plates) allow the anatomical shape required for the orthognathic corrections to be made to be obtained beforehand.

These implants or plates have particular features that are linked to various factors. The question of being made of pure titanium is not only linked to the question of mechanical strength, but also to the fact that the aforementioned pre-molded plates are biocompatible, with anatomical designs, low profile, having this wide range of rigid fixing system plates. As stated earlier, this generates less surgical trauma, faster recovery, lower risk of infection, less blood loss, and similarly smaller scars.

FIGS. 1 to 10 and 15 show Lefort type maxillary implants—plates (1)—which are segmented in the upper portions (2) and restructured in the central region (3). This central region (3) can have an indentation (3′) or be straight (3″). The indentations (3′) can be higher up (as shown in FIG. 1) or lower down (FIG. 3), or they can combine holes (4) in circular or oblong inclined surfaces, with indentations (3′) or straight surfaces (FIGS. 4 and 5); FIG. 6 shows plates similar to FIGS. 4 and 5, with the upper surface angled the other way. FIG. 15 shows plate (1) with the central part (3′) twisted (T) in opposite directions.

FIG. 7 shows a Lefort-type implant—plate (1)—with a straight, L-shaped lower part (5) with holes and an indentation (3′), while the upper part projects an inclined terminal to the right (6) with three holes.

FIG. 8 shows a Lefort-type implant—plate (1)—with the lower part inclined below the “X” axis (7) and three holes, from which projects a linear part (8) with parallel ribs (9), from which rises the upper part inclined to the left of the “Y” axis (10), with three holes.

FIG. 9 shows implant—plate (1)—of the Lefort type—with the lower part in an inverted “V” (11), having two holes on one side and three holes on the opposite side, from which an indentation (3′) is projected, followed by a vertical linear stretch (12), giving rise to an upper part (12′) with four holes at distal points horizontally and vertically.

FIG. 10 shows implant—plate (1)—of the Lefort type—very similar to that shown in FIG. 9, without the indentation (3′), which is replaced by a linear extension (3″).

FIG. 11 shows an implant—plate (1)—for chin advancement, which has a general “X” shape (13), with the lower part (14) having a smaller width than the upper part (15), both having holes at the ends of the “X” formed, in one piece and with an indentation (3′).

FIG. 12 shows an implant—plate (1)—for chin advancement, which has an “X” shape, with the lower part (16) having a smaller width than the upper part (17), both having holes at the ends of the formed “X”, in one piece and with an indentation (3′) the other way around in relation to the previous figure.

FIG. 13 shows an implant—plate (1)—for chin advancement, which has an “X” shape, with the lower part (18) being smaller in width than the upper part (19), both having holes at the ends of the formed “X”, in one piece and having a rectilinear central part (20).

FIG. 14 shows an implant—plate (1)—for chin advancement, in an “X” shape, with the lower part (21) having the same width as the upper part (22), both having holes at the ends of the formed “X”, in one piece and with the central part having an indentation (3′).

FIG. 16 shows an implant—plate (1)—for mandible advancement—with a base extension (23) with a slight undulation (24) between the two aligned holes, followed by a larger angled hole (25), from where the end bifurcates (26), which similarly have angled posterior holes (27).

FIG. 17 shows an implant—plate (1)—for mandible advancement—in a single and straight piece, where a base (28) with three holes is parallel to another base (29) with three other holes and said bases interconnected by a straight section (30).

FIG. 18 shows an implant—plate (1)—for mandible advancement—in a single and straight piece, similar to the previous one, but with the bases (28′) and (29′) projected to opposite sides in relation to the previous figure.

FIG. 19 shows an implant—plate (1)—for mandible advancement—in one piece, with a central sector (31), which projects on one side a terminal with an inclination (32) in relation to the longitudinal axis of the aforementioned central sector (31); this terminal with an inclination (32) has two holes which, like all the others, are threaded internally; the second terminal (33) projects at an opposite inclination to the previous one, and has two angled holes (34).

FIG. 20 shows an implant—plate (1)—for mandible advancement—in one piece, having a central rectilinear sector (35) with two holes on its right and a hole on the left of the angled type (36), projecting an inclined terminal (37) with extreme angled hole (38).

FIG. 21 illustrates the application of the invention's implants (plates) on an oral and maxillofacial model, in an example of the use of the Lefort type, where the plates are applied in oral and maxillofacial correction in the maxillary region.

FIG. 22 illustrates the application of the invention's implants (plates) on an oral and maxillofacial model, in an example of the use of the chin type, where the plates are applied in oral and maxillofacial correction in the chin region.

FIG. 23 illustrates the application of the invention's implants (plates) on an oral and maxillofacial model, in an example of use, the plates are applied in oral and maxillofacial correction in the mandibular-sagittal region.

According to the present invention, the implants—plates (1)—in all their versions are provided with reduced design—smaller and less thick—, in addition to having angled posterior holes, and having restructured central regions—with indentations (3′) or straight—, being also precast with these characteristics of reduced design, angled holes and restructured central regions. All implants or plates are supplied in osteosynthesis cases.

Therefore, the use of the plates of this invention means that, unlike traditional techniques, surgical accesses are minimal and with less tissue displacement, reducing surgical time and providing a faster and more satisfactory post-operative period. In other words, according to the invention, plates are presented with a reduced design, angled posterior holes, restructured central region, greater rigidity and pre-molded; these are some of the differentials offered by the new osteosynthesis cases with the plates presented here.

In other words, the invention establishes new ways of fixing the plates (1) through the reduced design, as already explained, the use of indentations and angled holes, making the operation endowed with all the facilities and improvements already listed, both for the patient and the professional.

1. An implant with adjustable plates for minimally invasive orthognathic surgery, comprising a range of titanium implants (plates) with internally threaded holes for use in minimally invasive orthognathic surgery, made up of chin-type plates and mandibular Lefort-type plates, Lefort-type maxillary plates and sagittal-type mandibular plates, all with a reduced design, which are segmented in the upper portion and restructured in the central region and pre-molded straight and “L” plates—also Lefort, these plates are intended for surgical correction procedures in the mandible, maxillary and chin regions; wherein the fact that the pre-molded plates are biocompatible, with anatomical designs, low profile, with this wide range of plates having a rigid fixation system, i.e. smaller and less thick—as well as having angled posterior holes, and having restructured central regions—with indentations or straight—, while still being pre-molded with these characteristics of reduced design, angled holes and restructured central regions.

2. The implant with adjustable plates for minimally invasive orthognathic surgery according to claim 1, wherein implants—plates—of the Lefort type—maxilla—, which are segmented in the upper portions and restructured in the central region, this central region being able to be provided with an indentation or straight; the indentations can be higher or lower, or combine holes in circular or oblong sloping surfaces, with indentations or straight surfaces; by having an upside-down angled upper surface.

3. The implant with adjustable plates for minimally invasive orthognathic surgery, according to claim 1, wherein an implant—plate—of the Lefort type—with a straight and “L” shaped lower part with holes and an indentation, while the upper part projects a terminal inclined to the right with three holes.

4. The implant with adjustable plates for minimally invasive orthognathic surgery, according to claim 1, wherein an implant—plate—of the Lefort type—with the lower part inclined below the “X” axis and three holes, from which a linear part with parallel ribs projects, from which the upper part inclined to the left of the “Y” axis, with three holes, rises.

5. The implant with adjustable plates for minimally invasive orthognathic surgery, according to claim 1, wherein an implant—plate—of the Lefort type—with an inverted “V” shaped lower part, having two holes on one side and three holes on the opposite side, from which an indentation projects, followed by a vertical linear section, giving rise to an upper part with four holes at distal points horizontally and vertically.

6. The implant with adjustable plates for minimally invasive orthognathic surgery, according to claim 1, wherein an implant—plate—of the Lefort type—in which the indentation is replaced by a linear extension.

7. The implant with adjustable plates for minimally invasive orthognathic surgery, according to claim 1, wherein an implant—plate—for chin advancement, which has the general conformation of an “X”, with the lower part having a smaller width than the upper part both having holes at the ends of the formed “X”, in one piece and with an indentation.

8. The implant with adjustable plates for minimally invasive orthognathic surgery, according to claim 1, wherein an implant—plate—for chin advancement, which has the conformation of an “X”, with the lower part having a smaller width than the upper part, both having holes at the ends of the formed “X”, in one piece and with an indentation unlike the previous one.

9. The implant with adjustable plates for minimally invasive orthognathic surgery, according to claim 1, wherein an implant—plate—for chin advancement, which has the conformation of an “X”, with the lower part having a smaller width than the upper part, both having holes at the ends of the formed “X”, in one piece and having the rectilinear central part.

10. The implant with adjustable plates for minimally invasive orthognathic surgery, according to claim 1, wherein an implant—plate—for chin advancement, which has the conformation of an “X”, with the lower part having an equal width to the upper part, both having holes at the ends of the formed “X”, in one piece and with the central part equipped with indentation.

11. The implant with adjustable plates for minimally invasive orthognathic surgery, according to claim 1, wherein an implant—plate—for mandible advancement—with a base extension with a slight undulation between the two aligned holes, followed by a larger angled hole, from where the end bifurcates, which similarly have angled posterior holes.

12. The implant with adjustable plates for minimally invasive orthognathic surgery, according to claim 1, wherein an implant—plate—for mandible advancement—in a single and straight piece, where a base with three holes is parallel to another base with three other holes and said bases interconnected by a straight section.

13. The implant with adjustable plates for minimally invasive orthognathic surgery, according to claim 1, wherein an implant—plate—for mandible advancement—in a single and straight piece, similar to the previous one, but with the bases and projected to opposite sides.

14. The implant with adjustable plates for minimally invasive orthognathic surgery, according to claim 1, wherein an implant—plate—for mandible advancement—in one piece, with a central sector, which projects on one side a terminal with an inclination in relation to the longitudinal axis of the aforementioned central sector; this terminal with an inclination has two holes; the second terminal projects at an opposite inclination to the previous one, and has two angled holes.

15. The implant with adjustable plates for minimally invasive orthognathic surgery, according to claim 1, wherein an implant—plate—for mandible advancement—in one piece, having a central rectilinear sector with two holes on its right and a hole on the left of the angled type, projecting an inclined terminal with extreme angled hole.

16. A method of using of the plates shown in claim 1, comprising characterized by application of the implants (plates) of the Lefort type, in oral and maxillofacial correction in the maxillary region.

17. The method according to claim 16, wherein the use of implants (plates) of the chin type, wherein the plates are applied in oral and maxillofacial correction in the chin region.

18. The method according to claim 16, wherein application of the implants (plates) in oral and maxillofacial correction in the mandibular—sagittal region.
